(19) United States
(12) Patent Application Publication
(10) Pub. No.: US 2024/0423891 A1
(43) Pub. Date: Dec. 26, 2024

(54) COMPOSITIONS AND METHODS FOR SKIN BARRIER IMPROVEMENT AND IRRITATION REDUCTION

(71) Applicant: Amyris, Inc., Emeryville, CA (US)

(72) Inventors: Debora MILLARD, Emeryville, CA (US); Daniel Thorn LEESON, Emeryville, CA (US); Yu Wen CHEN, Emeryville, CA (US); Kevin Lee DIETZEL, Emeryville, CA (US); Morgan TARDY, Emeryville, CA (US); Jessica KIZOVSKI, Emeryville, CA (US); Carol Lin TRAN, Emeryville, CA (US)

(21) Appl. No.: 18/336,533

(22) Filed: Jun. 16, 2023

(51) Int. Cl. A61K 8/49; A61K 8/31; A61Q 5/00; A61Q 19/00

(52) U.S. Cl. CPC A61K 8/4953; A61K 8/31; A61Q 5/002; A61Q 19/00

(57) ABSTRACT

Provided herein are cosmetic compositions for increasing the integrity of the skin or hair a subject or for reducing irritation of the skin of a subject and methods of using the cosmetic compositions. The cosmetic compositions contain a combination of a occlusive compound and an osmolyte compound. The occlusives of the combinations are squalane, hemisqualane, isosqualane, squalene, or isosqualene. The preferred osmolyte is ectoine. The cosmetics or pharmaceuticals may be used to increase the barrier of the skin or hair, increase the integrity of skin or hair, reduce inflammation of the skin, or prevent aging of the skin of a subject.

### FIELD OF THE INVENTION

The present disclosure relates to cosmetic compositions comprising combinations of occlusive compounds and osmolyte compounds and methods of using the cosmetic compositions to treat the skin or hair of a subject.

### BACKGROUND

Having primarily protective functions, skin and hair are exposed to a number of insults during the life of a subject. Typical insults include UV light, air pollutants, low humidity air, water, and a variety of harsh chemical agents used by the subject to wash or condition their hair or skin. These insults often cause immediate effects such as inflammation or irritation of the skin. Longer term effects of the insults include premature aging and loss of integrity of the skin and hair. Given the importance of the skin and hair in protecting subjects from external insults, there is a need for cosmetic compositions that improve the barrier function of the skin and hair, that reduce inflammation or irritation, and that support the structural integrity of skin and hair.

### SUMMARY OF THE INVENTION

Provided herein are cosmetic compositions for improving the barrier function and/or increasing the integrity of the skin of a subject. In addition, the invention provides cosmetic compositions that reduce inflammation and irritation of the skin of the subject. Further, certain embodiments support the health and integrity of the hair of the subject.

In one aspect, the invention provides for a cosmetic composition containing one or more occlusive compounds and one or more osmolyte compounds.

In an embodiment of the invention the one or more occlusive compounds are selected from squalane, hemisqualane, isosqualane, squalene, and isosqualene. In another embodiment of the invention the one or more osmolytes compounds are selected from ectoine, trimethylamine N-oxide (TMAO), dimethylsulfoniopropionate, and hydroxyectoine. In specific embodiment of the invention the one or more osmolyte compounds comprise ectoine. In a preferred embodiment of the invention the occlusive compound is squalane and the osmolyte is ectoine. In an embodiment of the invention squalane comprises from about 0.01% w/w to about 99.99% w/w of the cosmetic composition. In another embodiment of the invention the squalane comprises from about 1% w/w to about 20% w/w of the cosmetic composition. In other embodiments of the invention the ectoine comprises from about 0.01% w/w to about 99.99% w/w of the cosmetic composition. In further embodiments the ectoine comprises from about 1% w/w to about 7% w/w of the cosmetic composition. In other embodiments the cosmetic composition contains an active ingredient. In an embodiment the active ingredient is selected from retinol, nicotinamide riboside, niacinamide, and vitamin C. In additional embodiments the cosmetic composition contains one or more excipients selected from the group consisting of natural oils, synthetic oils, emulsifiers, emollients, lipids, humectants, moisturizers, sunscreens, binders, powders, conditioning agents, emulsion stabilizing salts, preservatives, chelating agents, sequestering agents, abrasives, pH adjusters, surfactants, perfumes, and colorings. In an embodiment the humectant is selected from alpha-hydroxy acids (AHA), salicylic acid, glycerin, glycerol, hyaluronic acid, panthenol, and sodium lactate. In another embodiment the cosmetic composition is packaged with instructions for use. In additional embodiments the instructions for use provide instructions for topical application to skin of a subject. In an embodiment the instructions for use provide instructions for topical application to hair of a subject.

In another aspect the invention provides for a method of improving the integrity of skin or hair of a subject involving administering an effective amount of the cosmetic composition described herein to the skin or hair of the subject. In an embodiment the cosmetic composition is administered to the skin or hair in an amount of from about 0.1 mg/cm² to about 10 mg/cm². In another embodiment the cosmetic composition is administered to the skin or hair in an amount of from about 1 mg/cm² to about 3 mg/cm². In an additional embodiment the cosmetic composition is administered at least daily. In further embodiments the cosmetic composition is administered at least twice daily.

In yet another aspect the invention provides for a method of reducing irritation in skin of a subject comprising administering an effective amount of the cosmetic composition described herein to the skin of the subject, thereby reducing irritation. In an embodiment the cosmetic composition is administered to the skin in an amount of from about 0.1 mg/cm² to about 10 mg/cm². In another embodiment the cosmetic composition is administered to the skin or hair in an amount of from about 1 mg/cm² to about 3 mg/cm². In an additional embodiment the cosmetic composition is administered at least daily. In further embodiments the cosmetic composition is administered at least twice daily.

### BRIEF DESCRIPTION OF THE DRAWINGS

FIG. 1 is a graphic that illustrates the four aspects of the cosmetic compositions described herein: occlusion, humectancy, lipid replenishment, and protein stabilization.

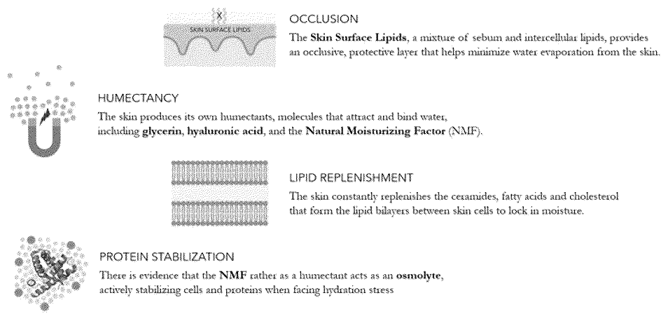

### DETAILED DESCRIPTION OF THE EMBODIMENTS

The invention provides cosmetic compositions useful for increasing the barrier/protective function and integrity of the skin and/or hair of a subject, wherein the composition is composed of a combination of a occlusive compound and an osmolyte compound. The invention also provides methods of treating the skin or hair of the subject involving the administration or application of the cosmetic composition. The cosmetic composition functions by replenishing the surface and cellular lipids of the skin (occlusion) and by stabilizing skin cells and proteins by maintaining cell volume and specific hydration (osmolytic stabilization). In addition, some embodiments of the invention also increase the moisture content of the skin or hair.

As used herein an “effective amount” means an amount necessary to at least partly attain the desired response, or to delay the onset or inhibit progression or halt altogether, the onset or progression of a particular symptom being treated. The amount varies depending upon the health and physical condition of the subject to be treated, the taxonomic group of subject to be treated, the degree of protection desired, the formulation of the composition, the assessment of the medical situation, and other relevant factors. It is expected that the amount will fall in a relatively broad range that can be determined through routine trials.

As used herein, “subject” or “patient” is an organism that is treated using one of the methods of the present disclosure. In an embodiment, the subject is a mammalian subject, such as a human or a domestic animal.

As used herein, the term “about” will be understood by persons of ordinary skill in the art and will vary to some extent on the context in which is used. If there are uses of the term which are not clear to persons of ordinary skill in the art given the context in which is used, “about” may mean up to plus or minus 20% of the particular term.

As used herein, the term “ectoine” refers to a compound having the following formula:

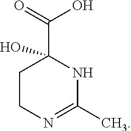 [SMILES: CC1=NCC[C@@](O)(C(=O)O)N1]

Ectoine is also referred to as (4S)-2-Methyl-3,4,5,6-tetrahydropyrimidine-4-carboxylic acid.

As used herein, “emollient” refers to any compound that will soften or smooth skin or hair following topical administration.

As used herein, “osmolyte” refers to any compound providing stabilization to cells, cellular components, and biological structures and molecules, including proteins, by affecting the properties of the biological fluids contained in or surrounding them and by controlling the hydration of cells and their components. Illustrative examples of an osmolytes are ectoine, trimethylamine N-oxide (TMAO), dimethylsulfoniopropionate, and hydroxyectoine.

As used herein, “squalane” refers to a compound having the following formula:

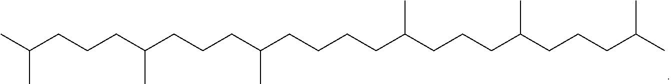 [SMILES: CC(C)CCCC(C)CCCC(C)CCCCC(C)CCCC(C)CCCC(C)C]

As used herein “hemisqualane” or “farnesane” refers to a compound having the following structure:

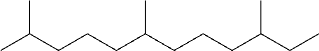

or a stereoisomer thereof.

As used herein, “iso-squalane” or “isosqualane” refers to a compound having the following formula:

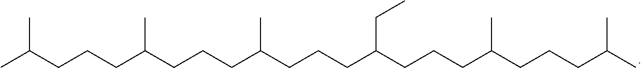 [SMILES: CCC(CCCC(C)CCCC(C)C)CCCC(C)CCCC(C)CCCC(C)C]

As used herein, “neosqualane” refers to a compound having the following formula:

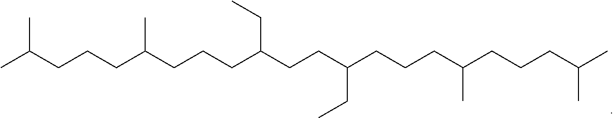 [SMILES: CCC(CCCC(C)CCCC(C)C)CCC(CC)CCCC(C)CCCC(C)C]

As used herein, “squalene” refers to a compound having the following formula:

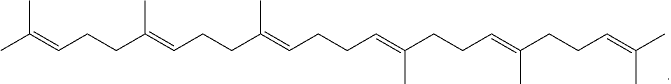 [SMILES: CC(C)=CCC/C(C)=C/CC/C(C)=C/CC/C=C(\C)CC/C=C(\C)CCC=C(C)C]

As used herein, “iso-squalene” or “isosqualene” refers to a compound having any one of the following structures:

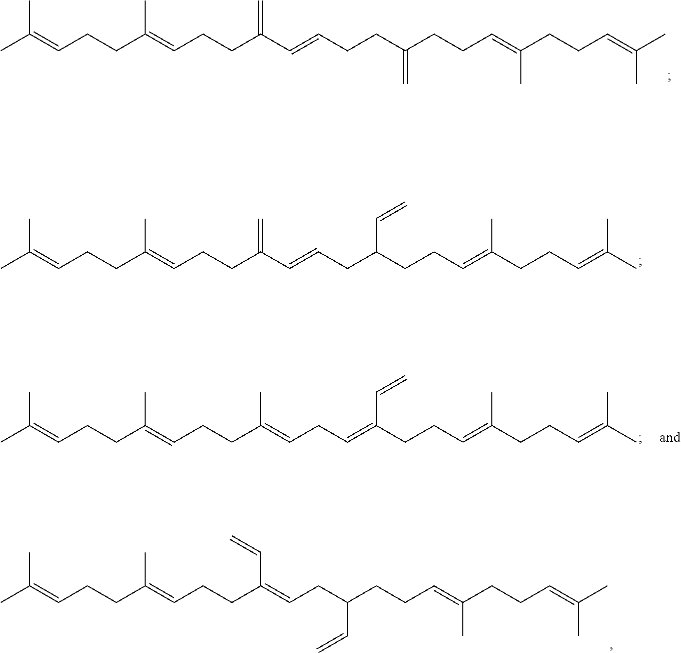

and any partially saturated derivative thereof.

As used herein, “occlusives” refer to any compound that acts as a barrier to retain moisture after topical administration to skin or hair.

As used herein, “humectant” refers to any compound that draws moisture into skin or hair following topical administration. Illustrative examples of humectants are alpha-hydroxy acids (AHA), salicylic acid, glycerin, glycerol, hyaluronic acid, panthenol, and sodium lactate.

As used herein, the term “cosmetic” or “cosmetic composition” means any substance or mixture intended to be placed in contact with various external parts of a subject's body (epidermis, hair systems, nails, lips, and external sex organs) or with the teeth and the mucous membranes of the oral cavity with the intent primarily to clean, perfume, or changing the appearance of the subject's body, teeth, or mucous membranes of the oral cavity.

Cosmetics of the invention may contain one or more excipients. Excipients include but are not limited to natural oils, synthetic oils, emulsifiers, emollients, lipids, humectants, moisturizers, binders, conditioning agents, emulsion stabilizing salts, preservatives, chelating agents, sequestering agents, abrasives, pH adjusters, surfactants, perfumes and colorings.

Cosmetics of the invention may contain one or more active agents. Active agents produce a beneficial biological response when applied to the skin or hair of a subject. Illustrative active agents include retinol, nicotinamide riboside, niacinamide, and vitamin C.

Compositions and cosmetics disclosed herein may comprise various amount per weight of the occlusive (squalane, hemisqualane, isosqualane, squalene, or isosqualene). Examples include 0.01% (w/w), 0.02% (w/w), 0.03% (w/w), 0.04% (w/w), 0.05% (w/w), 0.06% (w/w), 0.07% (w/w), 0.08% (w/w), 0.09% (w/w), 0.1% (w/w), 0.2% (w/w), 0.4% (w/w), 0.5% (w/w), 0.6% (w/w), 0.7% (w/w), 0.8% (w/w), 09. % (w/w), 1% (w/w), 2 (w/w), 3% (w/w), 4% (w/w), 5% (w/w), 6% (w/w), 7% (w/w), 8% (w/w), 9% (w/w), 10% (w/w), 15% (w/w), 20% (w/w), 25% (w/w), 30% (w/w), 35% (w/w), 40% (w/w), 45% (w/w), 50% (w/w), 55% (w/w), 60% (w/w), 65% (w/w), 70% (w/w), 75% (w/w), 80% (w/w), 85% (w/w), 86% (w/w), 87% (w/w), 88% (w/w), 89% (w/w), 90% (w/w), 91% (w/w), 92% (w/w), 93% (w/w), 94% (w/w), 95% (w/w), 96% (w/w), 97% (w/w), 98% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), and 99.9% (w/w) of the occlusive. Preferred embodiments comprise squalane as the occlusive.

Mixtures of squalane and another occlusive may be used to formulate the cosmetic compositions. A preferred mixture comprises squalane and hemisqualane. Squalane and hemisqualane may be mixed in various ratios based on weight. Illustrative example ratios of squalane to hemisqualane include 10 to 1, 5 to 1, 10 to 3, 5 to 2, 2 to 1, 5 to 3, 10 to 7, 5 to 4, 10 to 9, 1 to 1, 9 to 10, 4 to 5, 7 to 10, 3 to 5, 1 to 2, 2 to 5, 3 to 10, 1 to 5, and 1 to 10. A person of skill in the art would understand that intermediate ratios between these values could also be derived to form a cosmetic composition comprising squalane and hemisqualane. A preferred embodiment involves a mixture of squalane and hemisqualane mixed at a 1 to 1 ratio by weight.

Cosmetic compositions comprising an occlusive (for example squalane) may contain an amount of an osmolyte dictated by the use of the composition. The preferred osmolyte of the invention is ectoine. Osmolytes may be present in the compositions at a certain weight percentage (w/w). For example, osmolytes may comprise 0.01% (w/w), 0.02% (w/w), 0.03% (w/w), 0.04% (w/w), 0.05% (w/w), 0.06% (w/w), 0.07% (w/w), 0.08% (w/w), 0.09% (w/w), 0.10% (w/w), 0.11% (w/w), 0.12% (w/w), 0.13% (w/w), 0.14% (w/w), 0.15% (w/w), 0.16% (w/w), 0.17% (w/w), 0.18% (w/w), 0.19% (w/w), 0.20% (w/w), 0.25% (w/w), 0.30% (w/w), 0.35% (w/w), 0.40% (w/w), 0.45% (w/w), 0.50% (w/w), 0.55% (w/w), 0.60% (w/w), 0.65% (w/w), 0.70% (w/w), 0.75% (w/w), 0.80% (w/w), 0.85% (w/w), 0.90% (w/w), 0.95% (w/w), 1.0% (w/w), 1.1% (w/w), 1.2% (w/w), 1.3% (w/w), 1.4% (w/w), 1.5% (w/w), 1.6% (w/w), 1.7% (w/w), 1.8% (w/w), 1.9% (w/w), 2.0% (w/w), 2.1% (w/w), 2.2% (w/w), 2.3% (w/w), 2.4% (w/w), 2.5% (w/w), 2.6% (w/w), 2.7% (w/w), 2.8% (w/w), 2.9% (w/w), 3.0% (w/w), 3.5% (w/w), 4.0% (w/w), 4.5% (w/w), 5.0% (w/w), 5.5% (w/w), 6.0% (w/w), 6.5% (w/w), 7.0% (w/w), 7.5% (w/w), 8.0% (w/w), 8.5% (w/w), 9.0% (w/w), 9.5% (w/w), and 10% (w/w). In some embodiments, the maximum amount of an osmolyte comprising the composition will be limited by the solubility limit of the osmolyte in the formulation. In those cases, the maximum amount of osmolyte can be determined empirically.

As used herein, the term “cosmetic” or “cosmetic composition” means any substance or mixture intended to be placed in contact with various external parts of a subject's body (epidermis, hair systems, nails, lips, and external sex organs) or with the teeth and the mucous membranes of the oral cavity with the intent primarily to clean, perfume, or changing the appearance of the subject's body, teeth, or mucous membranes of the oral cavity.

Cosmetics of the invention may contain one or more excipients. Excipients include but are not limited to natural oils, synthetic oils, emulsifiers, emollients, lipids, humectants, moisturizers, binders, conditioning agents, emulsion stabilizing salts, preservatives, chelating agents, sequestering agents, abrasives, pH adjusters, surfactants, perfumes and colorings.

The invention provides for the use of combinations of squalane and ectoine as a cosmetic to facilitate the treatment of skin and hair. However, one skilled in the art will readily appreciate, embodiments of the cosmetic of the invention may include other combinations of occlusives and osmolytes in addition to squalane and ectoine. For example, other occlusive compounds that can be used in embodiments of the invention include: hemisqualane, isosqualane, squalene, isosqualene, beeswax, vegetable waxes, plant oils and butters high in oleic acid, lecithin, cocoa butter, and allantoin. In addition, other osmolytes that can be used in embodiments of the invention include: trimethylamine N-oxide (TMAO), dimethylsulfoniopropionate, and hydroxyectoine.

Examples of the cosmetically active agents include vitamins, such as vitamin B, vitamin C, tocopherols (vitamin E), tocopherol derivatives, tocotrienols, vitamin D, K and derivatives thereof, and suitable combinations thereof, for example.

Moisturizing compounds (humectants) may include glycerin, urea, methylurea, ethylurea, allantoin, lactates, sugars, methyl glucose ethers, sodium pyrrolidone carboxylic acid, sodium hyaluronate, panthenol, hyaluronic acid, α- and β-hydroxyl acids, such as glycolic acid, lactic acid, mandelic acid, or salicylic acid, or combinations of the suitable moisturizing compounds, for example.

The compositions containing the extracts may further comprise a penetration enhancer, such as tissue penetration enhancers, to enhance release and delivery of the active compound into tissues, such as, for example, skin, ocular, nasal. For these compositions, they might comprise both release enhancing agents of the present embodiment and tissue penetration enhancers. The tissue penetration enhancers may then enhance tissue absorption and penetration of the released active agent. The tissue penetration enhancers might be either in suspended solid or solubilized form in the compositions of the present embodiment.

Tissue penetration enhancers, such as suitable volatile organic solvents, include aliphatic, cycloaliphatic and/or aromatic-aliphatic alcohols, each of which is monohydric or polyhydric, alcohol/water mixtures, saturated and/or unsaturated fatty alcohols which each contains from about 8 to about 18 carbon atoms, saturated and/or unsaturated fatty acids which each contains from about 8 to about 18 carbon atoms and/or esters thereof and the like and mixtures thereof. Useful alcohols are those having from 1 to about 20 carbon atoms, e.g., ethanol, isopropyl alcohol, 1-butanol, 1-octanol, etc.

The compositions of the invention can be administered or applied in a variety of unit dosage forms relative to any combination of occlusive compound and osmolyte compound depending upon the method of administration or application. Dosages for typical occlusive and osmolytes are well known to those of skill in the art. Such dosages are typically advisory in nature and are adjusted depending on the particular therapeutic context, patient or skin tolerance, etc. The amount of occlusive and osmolyte in the combination adequate to accomplish this is defined as an “effective dose.” The dosage schedule and amounts effective for this use, i.e., the “administration regimen,” will depend upon a variety of factors. In calculating the administration regimen for skin, the mode of administration or application also is taken into consideration. The administration or application regimen must also take into consideration the pharmacokinetics, i.e., the occlusive's, osmolyte's, and the combination's rate of absorption, bioavailability, metabolism, clearance, and the like. (See, e.g., the latest Remington's; Egleton and Davis 1997 Peptides 18:1431-1439; Langer 1990 Science 249:1527-1533).

The compositions and cosmetics provided herein may be applied to any available skin area that is in need of treatment. In some embodiments the composition or cosmetic is applied to the skin area liberally such that the skin area is saturated with the composition or cosmetic. In other embodiments it would be preferable to apply a specific amount of the composition or cosmetic to a specific amount of skin area (for example 1 mg composition per 1 cm² skin area). The compositions and cosmetics may be applied at 0.1 mg/cm², 0.2 mg/cm², 0.3 mg/cm², 0.1 mg/cm², 0.4 mg/cm², 0.5 mg/cm², 0.6 mg/cm², 0.7 mg/cm², 0.8 mg/cm², 0.9 mg/cm², 1.0 mg/cm², 1.1 mg/cm², 1.2 mg/cm², 1.3 mg/cm², 1.4 mg/cm², 1.5 mg/cm², 1.6 mg/cm², 1.7 mg/cm², 1.8 mg/cm², 1.9 mg/cm², 2.0 mg/cm², 2.1 mg/cm², 2.2 mg/cm², 2.3 mg/cm², 2.4 mg/cm², 2.5 mg/cm², 2.6 mg/cm², 2.7 mg/cm², 2.8 mg/cm², 2.9 mg/cm², 3.0 mg/cm², 3.5 mg/cm², 4.0 mg/cm², 4.5 mg/cm², 5.0 mg/cm², 5.5 mg/cm², 6.0 mg/cm², 6.5 mg/cm², 7.0 mg/cm², 7.5 mg/cm², 8.0 mg/cm², 8.5 mg/cm², 9.0 mg/cm², 9.5 mg/cm², and 10.0 mg/cm². In preferred embodiments the composition or cosmetic is applied at from about 1 mg/cm² to about 3 mg/cm².

The compositions and cosmetics may be applied as needed to ameliorate the condition or symptom being treated. In embodiments the compositions and cosmetics are applied at least once daily. In preferred embodiments the compositions and cosmetics are applied at least twice daily.

The compositions provided herein may be tested or optimized through use of skin models. Appropriate skin models are well known in the art and have been described in U.S. Pat. Nos. 10,323,230; 10,150,265; 10,073,085; and 9,592,257, each of which is incorporated in their entirety herein. A preferred skin model is commercially available as EPISKIN, and has been described at least in U.S. Pat. No. 10,039,791. In a typical skin model, cultured cells representing the predominant cell types of the epidermis are cultured as flat sheets on a support. In a preferred method, a three-dimensional culture of the cells is grown on the membrane of a cell well insert. Such a configuration allows for the positioning of the external (outer layer) of the reconstructed skin to be exposed to the upper surface of the insert and the basal cell layer of the reconstructed skin to be exposed to the lower or membrane surface of the insert. After reconstitution, the cells are cultured under defined conditions in cell culture media whereupon the cells fully differentiate and form the reconstructed skin layer. The use of cell wells and cell well inserts allows for experiments testing the delivery or permeation of skin by various test samples. In this configuration, the upper chamber of the insert is referred to as the “donor well” which contains the donor fluid. The chamber of the well in which the insert sits is the “receptor well” which contains the receptor fluid. In a typical delivery and/or permeation experiment, a test sample is applied to the donor well—in other words the test sample is applied to the surface of the reconstructed skin which represents the outer layer of the skin. The insert is then cultured at physiological temperature (37° C.) in a tissue culture incubator to maintain proper temperature, CO₂, and pH. At appropriate intervals, the donor fluid containing the test sample is removed, the cell insert is washed, and the samples are collected. Typically, three samples can be collected: the donor fluid, the skin cell fraction, and the receptor fluid. Each fraction can be assayed using the appropriate assay needed to measure the amount of the test compound in each fraction. In the case of occlusives and osmolytes, the amount of each in each fraction can be determined by use of high-pressure liquid chromatography (HPLC). In HPLC, sample is loaded on an appropriate column and run with an appropriate mobile phase. Purified preparations of the occlusive and osmolyte are run as a standard. In this case, the compounds can be identified by their retention time on the column and its concentration or amount can be determined by quantitating the area under the peak and deriving the amount using an algorithm determined by running known amounts of the control standard occlusive or osmolyte sample. The amount of occlusive and osmolyte in the skin cell fraction is an indication of the ability of each to be delivered into the skin cells. The amount of occlusive and osmolyte in the receptor fluid is an indication of the amount of each that is able to permeate the entire skin layer.

### EXAMPLES

The studies in this example will evaluate the ability of formulations comprising a mixture of an osmolyte compound and an occlusive compound to improve various skin attributes related to moisturization, aging, skin irritation, or skin disorders (e.g., eczema).

Study formulations will be prepared containing various amounts by weight of an osmolyte compound and an occlusive compound in order to evaluate the effects of each combination of osmolyte compounds and occlusive compounds. Initial studies will focus on combinations where the osmolyte compound is ectoine and the occlusive compound is squalane. Study formulations containing squalane in an amount from 0.01% w/w to about 99.99% w/w and containing ectoine in an amount from 99.9% w/w to about 0.01% w/w will be prepared. Most study samples will contain squalane in an amount from about 1% w/w to about 20% w/w and ectoine in an amount from about 1% w/w to about 7% w/w. Control samples may contain just squalane, just ectoine, or just another inert material known to have no effect on the measured variables.

Suitable human subjects will be recruited for the studies. For studies related to aging and skin disorders, subjects will be recruited that have measurable signs of aging, for example the presence of wrinkles or age spots on the skin, or having the skin disorder, for example eczema. In other studies, healthy subjects will be subjected to a “challenge,” for example exposure to ultraviolet light in an amount to elicits a measurable response. Subjects will be selected using a set of inclusion and exclusion criteria. Examples of exclusion criteria for healthy subjects will include the presence of any disease state, especially those that effect the skin. Inclusion criteria for disease studies will include the presence of the disease.

In challenge studies, study formulations or control samples will be applied to the skin of healthy subjects prior to, during, or after the challenge. At various time points post-challenge, the treated skin of the subject will be evaluated for response to the challenge. The number of subjects evaluated will be determined in order to provide statistical significance to the results. In general, subject responses over time will be evaluated and data from various study formulation treated subjects will be compared to untreated subjects and/or subjects treated with control formulations.

In skin disorder studies, subjects will be treated with various study formulations or control samples. Various skin parameters that are indicative of the skin disorders will be measured over time (typically between two and twelve weeks) to determine if the study formulations provide any improvement in the various parameters being monitored (i.e., the parameters more closely resemble those seen in skin from younger subjects in aging studies (e.g., increased dermal thickness) or the parameters more closely resemble those in healthy subjects (e.g., reduced redness or flakiness).

In some studies, transient short-term benefits of the study formulations will be evaluated by taking measurements at periods between the time of treatment and twenty-four hours, after application of a study formulation or control sample.

In some studies, the parameters measured will relate to moisturization and the quality of the skin barrier. These parameters will be determined by measuring transepidermal water loss (TEWL), hydration (measured using electrical conductance (i.e., using a corneometer), or skin dryness (measured by expert grading). Parameters related to skin irritation including redness, burning, or stinging will be evaluated by expert grading or by instrumentation. Aging related parameters including lines and wrinkles, firmness, dermal thickness, sagging, skin texture, hyperpigmentation/age spots, evenness of tone, and skin elasticity will be measured by expert grading and/or instrumentation (e.g., clinical photography/imaging combined with quantitative image analysis.

In addition, subjects will be asked for their self-perception of any skin parameters or attributes being studied.

### Study Designs

### Long Term Moisturization Study

Study subjects having dry skin or impaired barrier function will be recruited. Study formulations or controls will be applied to the skin of the study subjects daily over a study period of from one to four weeks. Skin dryness and/or hydration will be measured daily.

### Short Term Hydration Study

Study subjects having poor skin hydration or impaired barrier function will be recruited. Study formulations or controls will be applied to the study subjects' skin once. Skin dryness and/or hydration will be measured at time zero and at several time points over an eight-hour time course.

### Challenge Study

Healthy study subjects will be recruited. The skin of the study subjects will be challenged with a patch application of a harsh surfactant (e.g., SLS for 24 hours) or ultraviolet light (UV) exposure or a bioactive agent known to cause irritation or dryness (e.g., retinol). In some studies, study formulations or controls will be applied prior to the challenge. In other studies, study formulations or controls will be applied after the challenge. In some studies, study formulation will continue to be applied daily for one or two weeks after the challenge. Attributes related to the challenge irritation will be monitored periodically after the challenge.

### Long Term Studies

Study subjects having attributes associated with aging will be recruited. Study formulations or controls will be applied periodically (typically daily) over a long period of time (typically for four to sixteen weeks). Various skin attributes associated with aging will be measured periodically over the course of the study.

What is claimed:

1. A cosmetic composition comprising one or more occlusive compounds and one or more osmolyte compounds.

2. The cosmetic composition of claim 1, wherein the one or more occlusive compounds are selected from squalane, hemisqualane, isosqualane, squalene, and isosqualene.

3. The cosmetic composition of claim 1, wherein the one or more osmolytes compounds are selected from ectoine, trimethylamine N-oxide (TMAO), dimethylsulfoniopropionate, and hydroxyectoine.

4. The cosmetic composition of claim 3, wherein the one or more osmolyte compounds comprise ectoine.

5. The cosmetic composition of claim 4, wherein the occlusive compound is squalane and the osmolyte is ectoine.

6. The cosmetic composition of claim 5, wherein squalane comprises from about 0.01% w/w to about 99.99% w/w of the cosmetic composition.

7. The cosmetic composition of claim 5, wherein the ectoine comprises from about 0.01% w/w to about 99.99% w/w of the cosmetic composition.

8. The cosmetic composition of claim 1 further comprising an active ingredient.

9. The cosmetic composition of claim 8, wherein the active ingredient is selected from retinol, nicotinamide riboside, niacinamide, and vitamin C.

10. The cosmetic composition of claim 1 further comprising one or more excipients selected from the group consisting of natural oils, synthetic oils, emulsifiers, emollients, lipids, humectants, moisturizers, sunscreens, binders, powders, conditioning agents, emulsion stabilizing salts, preservatives, chelating agents, sequestering agents, abrasives, pH adjusters, surfactants, perfumes, and colorings.

11. The cosmetic composition of claim 10, wherein the humectant is selected from alpha-hydroxy acids (AHA), salicylic acid, glycerin, glycerol, hyaluronic acid, panthenol, and sodium lactate.

12. The cosmetic composition of claim 1, wherein the cosmetic composition is packaged with instructions for use.

13. The cosmetic composition of claim 12, wherein the instructions for use provide instructions for topical application to skin and/or hair of a subject.

14. A method of improving the integrity of skin or hair of a subject comprising administering an effective amount of the cosmetic composition of claim 1 to the skin or hair of the subject.

15. The method of claim 14, wherein the cosmetic composition is administered to the skin or hair in an amount of from about 0.1 mg/cm² to about 10 mg/cm².

16. The method of claim 14, wherein the cosmetic composition is administered at least daily.

17. A method of reducing irritation in skin of a subject comprising administering an effective amount of the cosmetic composition of claim 1 to the skin of the subject, thereby reducing irritation.

18. The method of claim 17, wherein the cosmetic composition is administered to the skin in an amount of from about 0.1 mg/cm² to about 10 mg/cm².

19. The method of any one of claims 17, wherein the cosmetic composition is administered at least daily.
